(19) United States
(12) Patent Application Publication
(10) Pub. No.: US 2005/0096502 A1
(43) Pub. Date: May 5, 2005

(54) Robotic surgical device

(76) Inventor: Theodore M. Khalili, Brentwood, CA (US)

Correspondence Address: MORRISON & FOERSTER LLP, 755 PAGE MILL RD, PALO ALTO, CA, 94304-1018, US

(21) Appl. No.: 10/697,584

(22) Filed: Oct. 29, 2003

(51) Int. Cl. A61B 1/00

(52) U.S. Cl. 600/106; 600/104; 600/129

(57) ABSTRACT

Described herein is a robotic surgical device configured for performing minimally invasive surgical procedures. The robotic surgical device comprises an elongated body for insertion into a patient's body through a small incision. In one variation, the elongated body houses a plurality of robotic arms. Once the distal portion of the elongated body is inserted into the patient body, the operator may then deploy the plurality of robotic arms to perform surgical procedures within the patient's body. An image detector may be positioned at the distal portion of the elongated body or on one of the robotic arms to provide visual feedback to the operator of the device. In another variation, each of the robotic arms comprises two or more joints, allowing the operator to maneuver the robotic arms in a coordinated manner within a region around the distal end of the device.

### FIELD OF THE INVENTION

The present invention generally relates to devices and methods for performing minimally invasive surgery. In particular, the invention relates to robotic devices designed for performing minimally invasive surgery.

### DESCRIPTION OF RELATED ART

Minimally invasive surgery has become more and more common nowadays. The surgical procedure is performed through multiple small incisions on the patient's body to minimize tissue damage and blood loss during surgery. The success of various minimally invasive surgical procedures in decreasing patient pain and improve recovering time has driven the trend to develop devices and procedures that would allow less invasive surgical procedure to be performed.

Most of the minimally invasive surgical procedures are performed with the help of a small endoscopic camera and several long, thin, and rigid instruments. The camera and the instruments are inserted into the patient's body through natural body openings or small artificial incisions. For example, in a typical cholecystectomy (gallbladder removal) procedure, a needle is inserted into the abdomen and insufflation is achieved by delivery of CO₂ gas into the abdomen. An endoscopic camera is inserted into the abdomen through an incision around the navel region, and additional instruments are inserted into the abdomen through incisions made on the right and left side of the abdomen.

The instrument typically comprises a long and rigid rod with a mechanical tool, such as a forceps or scissors, attached at the distal end of the rod. Mechanical connections are provided within the rod so that the surgeon may operate the tool from the distal end of the instrument through attachments at the proximal end of the instrument. With several of these long and rigid instruments, the surgeon proceeds to dissect out the gallbladder from its surrounding tissues, and seal off the blood vessels. Rods with various tools, such as forceps, scissors, and coagulator, may be introduced through the various incisions that are made on the abdomen to complete the necessary tasks. Finally the gallbladder is cut and removed from the body.

As discussed earlier, minimally invasive surgery causes significantly less trauma to the patient's body and thus improves patient recovery time. However, the technique itself also introduces other disadvantages for the surgeon. These complications include difficult hand-eye coordination and significant decrease in tactile perception. In addition, because the elongated instruments are inserted into the body from various directions, they tend to be difficult to handle. Further more, the confined space within the abdomen makes it even harder to maneuver the tools at the distal end of the instrument through the long and rigid rods.

Recently, robotic devices have been introduced to address some of these difficulties by improving dexterity and range of motion. However, the typical robotic surgical instrument still is made up of elongated rods each with a single tool attached at the distal end of the rod. Thus, a typical surgery still requires multiple incision sites in order to introduce all the necessary instruments into the patient's body. In addition, each instrument is connected to a separate electric-mechanical support device and requires a separate holder or frame to hold it in place. To prepare the multiple instruments and their corresponding electromechanical supporting devices for surgery increases the complexity of the pre-surgical set-up process and also increases the prep time for the surgery. In addition, during surgery, each instrument has to be inserted through a separate incision and then carefully positioned within the patient's body so that the different instruments may function in a coordinated manner. When computers are used to assist the surgeon in controlling the robotic devices, calibration and alignment of the various devices may be needed before each surgery. These additional processes tend to increase the complexity of the surgical procedure and extend the time needed to complete the procedure.

Various robotic devices have been previously devised for performing surgical procedures. Examples of such devices are disclosed in U.S. Patent Application, Publication No. 2002/0111713 A1, entitled “AUTOMATED ENDOSCOPE SYSTEM FOR OPTIMAL POSITIONING” published Aug. 15, 2002; U.S. Patent Application, Publication No. 2003/0083650 A1, entitled’ METHOD AND APPARATUS FOR PERFORMING MINIMALLY INVASIVE CARDIAC PROCEDURES” published May 1, 2003; U.S. Patent Application, Publication No. 2003/0083651 A1, entitled “METHOD AND APPARATUS FOR PERFORMING MINIMALLY INVASIVE CARDIAC PROCEDURES, published May 1, 2003; U.S. Pat. No. 4,943,296, titled “ROBOT FOR SURGICAL OPERATION issued to Funakubo et al., dated Jul. 24, 1990; U.S. Pat. No. 5,086,401, titled “IMAGE-DIRECTED ROBOTIC SYSTEM FOR PRECISE ROBOTIC SURGERY INCLUDING REDUNDANT CONSISTENCY CHECKING, issued to Glassman et al., dated Feb. 4, 1992; U.S. Pat. No. 5,996,346, titled “ELECTRICALLY ACTIVATED MULTI-JOINTED MANIPULATOR, issued to Maynard, dated Dec. 7, 1999; U.S. Pat. No. 6,102,850, titled “MEDICAL ROBOTIC SYSTEM” issued to Wang et al., dated Aug. 15, 2000; U.S. Pat. No. 6,231,565, titled “ROBOTIC ARM DLUS FOR PERFORMING SURGICAL TASKS” issued to Tovey et al., dated May 15, 2001; U.S. Pat. No. 6,398,726, titled “STABILIZER FOR ROBOTIC BEATING-HEART SURGERY” issued to Ramans et al., dated Jun. 4, 2002; U.S. Pat. No. 6,436,107, titled “METHOD AND APPARATUS FOR PERFORMING MINIMALLY INVASIVE SURGICAL PROCEDURES issued to Wang et al., dated Aug. 20, 2002; U.S. Pat. No. 6,447,443, titled “METHOD FOR ORGAN POSITIONING AND STABILIZATION issued to Keogh et al., dated Sep. 10, 2002; U.S. Pat. No. 6,470,236, titled “SYSTEM AND METHOD FOR CONTROLLING MASTER AND SLAVE MANIPULATOR issued to Ohtsuki, dated Oct. 22, 2002; and U.S. Pat. No. 6,554,844, titled “SURGICAL INSTRUMENT” issued to Lee et al., dated Apr. 29, 2003; each of which is incorporated herein by reference in its entirety. As seen in these examples, most of the existing devices require the introduction of multiple instruments into the patient's body for the procedure. In addition, the instruments usually are placed at multiple locations around the patient's body to complete the surgical procedure.

Therefore, an integrated device that allows simple deployment of multiple surgical tools inside a human body, thus, minimizing surgical trauma to the patient and decreasing the complexity involved in operating the surgical instruments, may provide substantial medical and economical benefits.

### SUMMARY OF THE INVENTION

Described herein is a robotic device for deploying and utilizing multiple surgical tools inside a patient. In one variation, the device comprises an elongated body where the distal end of the body is configured for insertion into a patient's body. The distal end of the elongated body houses a plurality of robotic arms. These robotic arms are configured for deployment inside a patient's body to provide surgical intervention. For example, two or more robotic arms may extend from the distal end of the device body. Each of the arms may comprise of two or more joints such that different arms may approach the same target tissue at a different angles or from a different direction. One or more tools may be attached to the distal end of each arm. An optional image detector or camera may be placed at the distal end of the elongated device body. Alternatively, the image detector may be placed at the distal end of an arm. In other variations, image detectors, sensors and surgical tools may also be placed along the length of the robotic arms, or at the distal portion of the device body.

A specific variation of the described device involves a robotic system made up of a single elongated arm having robotic arms and an optical viewing device such that but a single incision is necessary for carrying out a specific procedure.

A controller may be connected to the proximal end of the elongated device body. For example, an electronic controller with a monitor may be directly connected to the proximal end of the device body to allow the surgeon to control the robotic arms. Alternatively, an interface may be provided at the proximal end of the device body to allow a controlling unit to communicate with the device.

In one variation, the device comprises an elongated tubular body with an image detector positioned at the distal end of the tubular body. The distal portion of the tubular body has three chambers. Each of the chambers houses a separate robotic arm. The robotic arms extend outside the tubular body when deployed. Each of the robotic arms comprises three separate joints. The joints allow the three robotic arms to approach a predefined region from a different direction and with a different angle of approach. In an exemplary deployment, the first arm approaches the target tissue from the right side at an angle, and the second arm approaches the target tissue from the left side at an angle, and the third arm approaches the target tissue from the front of the tissue at an angle slightly above the target tissue. In this particular example, the distal end of the first arm has a bipolar forceps attached to it; the distal end of the second arm has a scissor; and the distal end of the third arm carries a vascular clip dispenser/applicator.

The integrated robotic surgical device allows the surgeon to introduce multiple surgical tools through a single incision. Once the distal end of the surgical device is placed inside the patient, the plurality of robotic arms is deployed to perform the surgical intervention. This integrated surgical device may also allow surgeons to perform intervention with techniques that are previously difficult to accomplish. For example, in situation where it is desirable for the surgeon to approach the target tissue from one direction, it would be difficult to accomplish with traditional laparoscopic techniques.

The integrated device permits the surgeon to perform laparoscopy surgery with fewer incisions. In some cases, the surgery may be accomplished with only one incision. For example, the integrated robotic surgical device may carry all the necessary tools and supplies to complete a surgical procedure. Alternatively, additional tools or supplies may be introduced through the same incision. In addition, the integrated robotic surgical device may allow the surgeon to perform surgery through natural openings in the human body. For example, surgery in the patient's stomach or intestine may be completed without a need for first making an incision.

Methods for utilizing a multi-arm robotic surgical device in performing minimally invasive surgical procedures are also contemplated. In one variation, the method comprises introducing a multi-arm surgical robot through a single incision and allowing the robotics arms to expand laterally such that the arms may approach the target issues from multiple direction/angles. The surgeon, through a control interface, maneuvers the robotic arms to complete the necessary surgical tasks.

The ability to introduce multiple robotic arms through a signal incision and having the plurality of arms function in a coordinated manner to accomplish a surgical task inside a patient's body may minimize trauma to patient, decrease pre-surgical prep time, and reduced the time necessary to accomplish the surgery. As the consequence, these benefits may reduce patient recovery time, improve procedure accuracy, and decrease overall cost of the procedure.

### BRIEF DESCRIPTION OF THE DRAWING

In the accompanying drawings, reference characters refer to the same parts throughout the different views. The drawings are intended for illustrating some of the principles of the robotic surgical device and are not intended to limit the description in any way. The drawings are not necessarily to scale, emphasis instead being placed upon illustrating the depicted principles in a clear manner.

FIG. 1A illustrates the distal portion of one variation of a robotic surgical device. In this variation, the body of the device houses three robotic arms and an image detector is positioned at the distal end of the elongated device body.

FIG. 1B illustrates the robotic surgical device shown in FIG. 1A, with two of its robotic arms deployed and a third robotic arm partially deployed.

FIG. 1C illustrates the robotic surgical device shown in FIG. 1A, with all three of its robotic arms deployed. The distal ends of the robotic arms are shown pointing at the same target area.

FIG. 1D is a top view of a robotic surgical device illustrating some of the possible ranges of motion that may be achieved by the robotic arm.

FIG. 2 illustrates one variation of the robotic surgical device having a tapered end at the distal portion of the elongated device body to facilitate insertion of the device into a patient's body.

FIG. 3 shows another example of the robotic surgical device having an interface at the proximal end of the device for communicating with a controller and for receiving power supply. In this variation, the distal portion of the elongated device body may rotate relative to the proximal portion of the body.

FIG. 4 illustrates another variation of the robotic surgical device where the distal portion of the device housing the robotic arms may be detached and replaced with a distal portion having a different set of surgical tools.

FIG. 5 illustrates an alternative design, where the device body comprises a conduit for supporting multiple robotic arms. In this particular variation, a camera is provided at the distal end of the device, and the device body has three channels for supporting three separate robotic arms.

FIG. 6 illustrates an optional feature of the robotic surgical device where the surgical tools may be detached from the distal end of the robotic arm and replaced with a different surgical tool.

FIG. 7A illustrates another variation of the robotic surgical device. In this variation, the camera is supported on a robotic arm that can be extended from the elongated body of the surgical device.

FIG. 7B illustrates another variation of the robotic surgical device where a conical shaped balloon is inflated at the distal end of the device.

FIG. 8 shows another variation of the robotic surgical device having an oval cross-section and two robotic arms which can be maneuvered to move in multiple directions. This variation of the device also has an image detector and a illuminating light source connected to the distal end of the elongated surgical device body.

FIG. 9A illustrates another variation of the robotic surgical device having robotic arms that are capable of axial rotation, and multiple segments of its arm are retractable.

FIG. 9B is a side view of the robotic surgical device shown if FIG. 9A.

FIG. 10A illustrates one example of a joint having two degrees of freedom, which is capable of both yaw and pitch motions.

FIG. 10B is a side view of a robotic arm implementing two joints, one joint having two degrees of motion and the other with only one degree of motion. The Robotic arm also supports an adapter for replacing the attached surgical tool.

FIG. 11 illustrates another approach to allow the attached robotic arms in the robotic surgical device to deploy in a lateral/radial direction. A top view of the device is shown.

FIG. 12A illustrates another variation of the robotic surgical device having robotics arms that are foldable for compact storage within the distal portion of the elongated body of the device. A top view of the device is shown.

FIG. 12B illustrate an alternative design of the robotic surgical device shown in FIG. 12A, where torsional motion is at the forearms of the robotic device.

FIG. 13A shown another design variation of the robotic surgical device in a closed position where the leaflets at the distal end of the device cover and protect the robotic arms.

FIG. 13B illustrates the robotic surgical device shown in FIG. 13A with all three of its robotic arms deployed.

FIG. 13C illustrate another variation of a robotic arm that is attached to a leaflet on a robotic surgical device. The robotic arm is shown to have the capability to move laterally in relation to the length of the leaflet.

FIG. 14A is the side view of another variation of a leaflet where extra space is provided under the leaflet for housing the robotic arm.

FIG. 14B illustrate a robotic surgical device, implementing the dome shaped leaflet shown in FIG. 14A, with all its leaflets in a close position.

FIG. 15 shows another approach to implement a robotic arm on the robotic surgical device.

FIG. 16 shows the front view of another variation of the robotic device where the robotic arms are connected to the distal end of the elongated main body of the device, and leaflets are provide to protect the robotic arm when the device is in the retracted position. The device is shown with all four of its robotic arms deployed. A camera is located between the four robotic arms.

### DESCRIPTION OF THE INVENTION

Before describing the present invention, it is to be understood that unless otherwise indicated this invention need not be limited to a device for performing surgical procedures. Surgical procedures are used herein as examples. It is under stood that some variation of the invention may be applied to various tasks where it would be desirable to deploy multiple robotic arms inside a mammalian body through a single incision. For example, the device may be utilized to accomplish a diagnostic task, such as taking physical or chemical measurements, or extracting a tissue sample from inside the patient's body.

Laparoscopic surgeries, such as cholecystectomy, are used herein as example applications to illustrate the functionality of the different aspects of the invention disclosed herein. It will be understood that embodiments of the present invention may be applied in a variety of minimally invasive surgical procedures and need not be limited to laparoscopic surgery. For example, in addition to other laparoscopic surgeries, such as laparoscopic appendectomy and laparoscopic colectomy, variations of the device may be implemented for arthroscopic surgery, endoscopic surgery, and for performing surgery in the thoracic or cranial cavities.

Surgical tools, such as scissors, coagulator, and forceps are used herein to illustrate the functionality of different aspects of the innovation disclosed herein. It will be understood that embodiments of the present invention are not limited to conventional surgical tools. The robotic arms may be implemented with various other mechanical or electrical tools, and various detectors or emitters. In addition, one or more of the robotic arms may be used to deliver and/or dispense surgical supplies (e.g., a vascular clip, or a dispenser housing multiple vascular clips), or for carrying other devices for delivering medical intervention.

It must also be noted that, as used in this specification and the appended claims, the singular forms “a,” “an” and “the” include plural referents unless the context clearly dictates otherwise. Thus, for example, the term “a camera” is intended to mean a single camera or a combination of cameras, “a liquid” is intended to mean one or more liquids, or a mixture thereof.

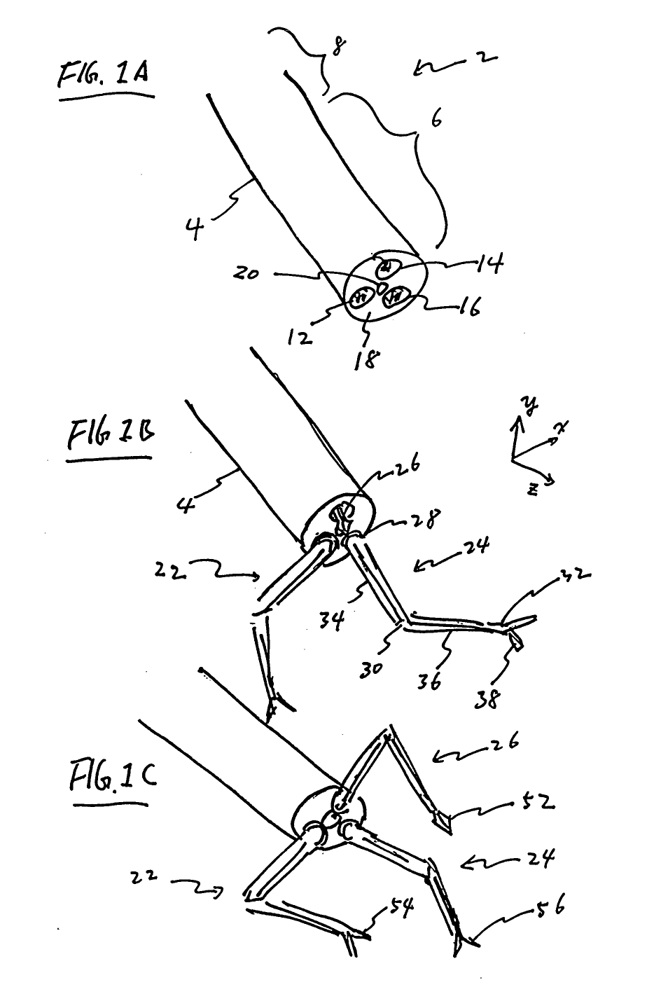

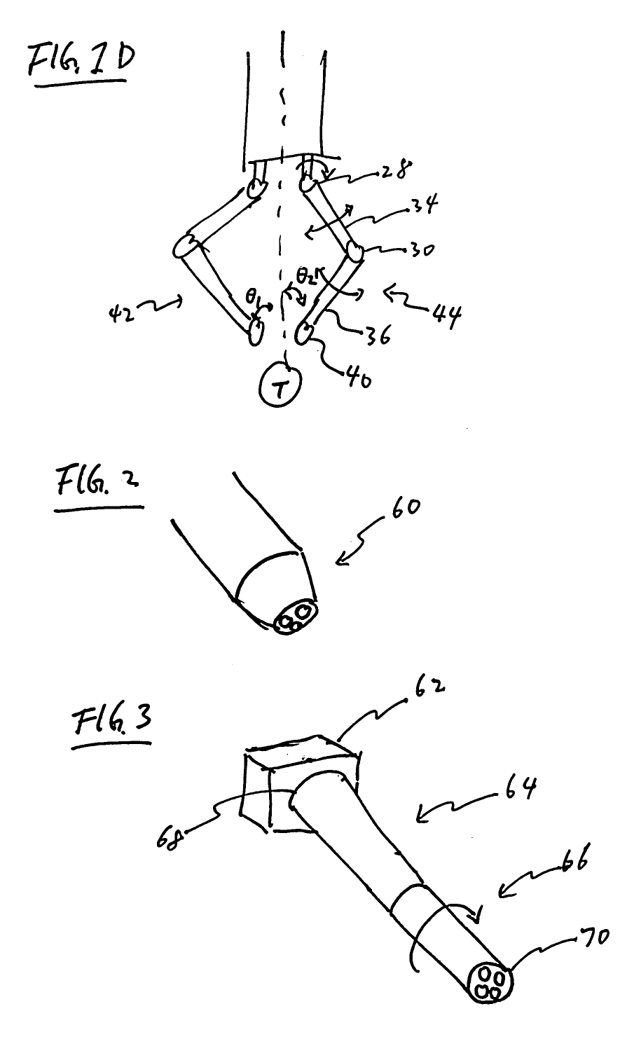

Referring to FIG. 1, one particular design variation of a multi-arm robotic surgical device 2 is shown. The device in this variation comprises an elongated body 4 with a circular cross section. The elongated body 4 may also be configured with other cross-sectional shape (e.g., oval, square, rectangular, pentagon, octagon, etc.). The distal portion 6 of the elongated body is configured for insertion into a patient's body through an incision or a natural orifice. The elongated body 4 may be rigid, flexible, or partially flexible depending on the particular application. For example, for laprascopic surgery, it may be desirable to have a rigid elongated body. For insertion into a patient's stomach, the distal section 6 of the elongated body may be rigid, and the proximal section 8 may be flexible so that it can be easily inserted down the esophagus. A plurality of robotic arms is configured for deployment from the distal portion 6 of the elongated body 4.

In this variation, the robotic arms are house within three chambers 12, 14, 16 at the distal portion of the elongated body 4. As seen in FIG. 1A, opening at the distal end 18 of the elongated body 4 allow the robotic arms to deploy from inside the elongated body 4. A forth chamber 20 houses an image detection device. The image detection device (i.e., image detector) may be a camera (e.g., a CCD camera, or an infrared camera), an optical detector, ultrasound detector, or a light sensor array. Alternatively, the chamber 20 may house an optical fiber, allowing light/image capture at the distal end 18 of the elongated body 4 to be directed to the proximal end of the body where an image detector may be implemented to capture the image. Optical lenses may be implemented such that the operator of the device may directly observe actions taking place at the distal end of the device directly. A light source (e.g., high intensity LED) may be utilized to provide illumination. The light source may share the same housing as the image detector. Alternatively, the light source may occupy its own chamber or be attached to the distal portion 6 of the elongated body 4.

An actuator or motor may be implemented for deploying the robotic arms. In one variation, each of the robotic arms is connected to an actuator for extending and retracting the distal sections of the robotic arm in and out of the chamber. Alternatively, a single displacement device is coupled to all three of the robotic arm and may extend and retrieve all three arms at the same time. In another variation, mechanical linkage is provided within the elongated body 4 such that the surgeon may deploy the robotic arms from the proximal end of the elongated body through a mechanical actuator or direct excretion of physical force.

FIG. 1B shows two of the robotic arms 22, 24 fully deployed, and a third robotic arm 26 partially deployed. In this variation, each arm comprises two primary joints. A first joint, the shoulder joint 28, may roll along a Z-axis that is parallel to the length of elongated body 4. In addition, the shoulder joint 28 may also allow a pitch movement, allowing the rear-arm 34 to move out of the Z-axis after the arm is deployed outside of its chamber. A second joint, the elbow joint 30, may allow the forearm 36 to rotate in relation to the rear-arm 34. A tool 38 or apparatus may be attached directly to the distal end of the forearm 36. However, in this variation, a third joint, the wrist joint 32, is provided. A surgical tool 38 or device may be attached to the wrist joint. The wrist joint 32 may provide pitch, yaw, and roll, three degrees of freedom. Alternatively, additional arm sections may be attached to the wrist joint 32 to extend the length of the arm. Additional arm section and joints may also be provided to further extend the length and maneuverability of the overall robotic arm. As one of ordinary skill in the art would appreciate, joints with different degrees of freedom may be implemented along the length of the robotic arm depending of the particular task the robotic arm is designed to perform.

Motors, actuator, or other displacement device may be implemented within each joint or along the length of the robotic arm to provide the mechanism to rotate each section of the arm. Alternatively, pulley systems may be implemented with displacement devices positioned within the elongated body 3 or at the proximal end of the elongated body to drive the motions of the arms.

Examples of various robotic assemblies, mechanical joints, and displacement mechanisms are disclosed in U.S. Patent Application, Publication No. 2002/0173700 A1, entitled “MICRO ROBOT” published Nov. 21, 2002; U.S. Patent Application, Publication No. 2003/0017032 A1, entitled “FLEXIBLE TOOL FOR HANDLING SMALL OBJECTS” published Jan. 23, 2003; U.S. Patent Application, Publication No. 2003/0180697A1, entitled “MULTI-DEGREE OF FREEDOM TELEROBOTIC SYSTEM FOR MICRO ASSEMBLY” published Sep. 25, 2003; U.S. Pat. No. 4,782,258, titled “HYBRID ELECTRO—PNEUMATIC ROBOT JOINT ACTUATOR” issued to Petrosky, dated Nov. 1, 1988; U.S. Pat. No. 4,822,238, titled “ROBOTIC ARM” issued to Kwech, dated Apr. 18, 1989; U.S. Pat. No. 4,946,421, titled “ROBOT CABLE-COMPLAINT DEVICES” issued to Kerley, Jr., dated Aug. 7, 1990; U.S. Pat. No. 5,113,117, titled “MINIATURE ELECTRICAL AND MECHANICAL STRUCTURES USEFUL FOR CONSTRUCTING MINIATURE ROBOTS” issued to Brooks et al., dated May 12, 1992; U.S. Pat. No. 5,136,201, titled “PIEZOELECTRIC ROBOTIC ARTICULATION” issued to Culp, dated Aug. 4, 1992; U.S. Pat. No. 5,157,316, titled “ROBOTIC JOINT MOVEMENT DEVICE” issued to Glovier, dated Oct. 20, 1992; U.S. Pat. No. 5,214,727, titled “ELECTROSTATIC MICROACTUATOR” issued to Carr et al., dated May 25, 1993; U.S. Pat. No. 5,245,885, titled “BLADDER OPERATED ROBOTIC JOINT issued to Robertson, dated Sep. 21, 1993; U.S. Pat. No. 5,265,667, titled “ROBOTIC ARM FOR SERVICING NUCLEAR STEAM GENERATORS” issued to Lester, II et al., dated Nov. 30, 1993; U.S. Pat. No. 5,293,094, titled “MINIATURE ACTUATOR” issued to Flynn et al., dated Mar. 8, 1994; U.S. Pat. No. 5,318,471, titled “ROBOTIC JOINT MOVEMENT DEVICE” issued to Glovier, dated Jun. 7, 1994; U.S. Pat. No. 5,327,033, titled “MICROMECHANICAL MAGNETIC DEVICES” issued to Guckel et al., dated Jul. 5, 1994; U.S. Pat. No. 5,331,232, titled “ON-THE-FLY POSITION CALIBRATION OF A ROBOTIC ARM” issued to Moy et al, dated Jul. 19, 1994; U.S. Pat. No. 5,357,807, titled “MICROMACHINED DIFFERENTIAL PRESSURE TRANSDUCERS” issued to Guckel et al., dated Oct. 25, 1994; U.S. Pat. No. 5,528,955, titled “FIVE AXIS DIRECT-DRIVE MINI—ROBOT HAVING FIFTH ACTUATOR LOCATED AT NON-ADJACENT JOINT” issued to Hannaford et al., dated Jun. 25, 1996; U.S. Pat. No. 5,778,730, titled “ROBOTIC JOINT USING METALLIC BANDS” issued to Solomon et al., dated Jul. 14, 1998; U.S. Pat. No. 6,256,134 B1, titled “MICROELECTROMECHANICAL DEVICES INCLUDING ROTATING PLATES AND RELATED METHODS” issued to Dhuler et al., dated Jul. 3, 2001; U.S. Pat. No. 6,374,982 B1, titled “ROBOTICS FOR TRANSPORTING CONTAINERS AND OBJECTS WITHIN AN AUTOMATED ANALYTICAL INSTRUMENT AND SERVICE TOOL FOR SERVICING ROBOTICS issued to Cohen et al., dated Apr. 23, 2002; U.S. Pat. No. 6,428,266 B1, titled “DIRECT DRIVEN ROBOT” issued to Solomon et al., dated Aug. 6, 2002; U.S. Pat. No. 6,430,475 B1, titled “PRESSURE-DISTRIBUTED SENSOR FOR CONTROLLING MULTI-JOINTED NURSING ROBOT” issued to Okamoto et al., dated Aug. 6, 2003; and U.S. Pat. No. 6,454,624 B1, titled “ROBOTIC TOY WITH POSABLE JOINTS” issued to Duff et al., Sep. 24, 2002; each of which is incorporated herein by reference in its entirety.

A computer may be implemented for controlling the various motors and actuators in the device so that the robotic arms may move in a coordinated manner. Sensors (e.g., pressure sensors, displacement sensor, or motion sensors, etc.) may be implemented within the robotic arm to provide feedback to the controlling computer. For example the displacement sensor may be placed within the elbow 30 to measure the amount of rotation of the forearm 36 relative to the rear-arm 34.

FIG. 1C shows the device with all three of its robotic arms 22, 24, 26 deployed. The three arms 22, 24, 26 are shown in an expanded position, where the three arms expends radially form the Z-axis of the device, and the tools are pointing toward a target region. As shown in FIG. 1D, a top view of the device illustrates the rotation of the shoulder joint 28 which allows the rear-arms 34 to expand radially from the Z-axis, and the elbow 30 allows the forearm 36 to rotate the distal end 40 of the forearm toward the Z-axis. The right forearm 42 is shown pointing toward the target region at an angle theta 1, and the left forearm 44 is shown pointing toward the target region at an angle theta 2. This configuration may allow the device to deploy multiple arms from a confined space and then allowing the arms to direct tools located at the distal end of each arm 40 into a given region from various directions.

FIG. 1C also illustrates various tools 52, 54, 56 attached to the distal end of each arm. Although one or more tools may be attached to the distal end of each arm, in this example, one tool per arm is shown. The right arm carries a forceps 52, the left arm carries a scissors 54, and the top arm carries a coagulator 56. A motor located in the forearm drives the forceps through mechanical interconnections for opening and closing the forceps. A pressure sensor may be implemented for measuring the amount of the pressure being applied by the forceps. The motor may be controlled by a controller that is connected to the device either directly or indirectly. The surgeon may then control the forceps through the controller. Alternatively, the forceps extends from an enclosure housing an actuator, which closes and expends the distal end of the forceps, and the proximal end of the enclosure is connected to the wrist of the right arm. A scissors 56 is connected to the wrist on the left arm, and a motor is provided in the left forearm to provide the mechanical force for closing and expanding the scissors. As one of ordinary skilled in the art would appreciate, various other configurations may be implemented for controlling the opening and closing of the scissors. A coagulator is connected directly to the distal end of the top arm 26. Electrical connection is provided such that electrical power may be provided through an electrical interface located at the proximal end of the device to provide the necessary electrical power to drive the coagulator.

To facilitate the insertion of the device into a patient's body, the distal end 60 of the device may be tapered, as shown in FIG. 2, to minimize abrasion caused by the edges at the distal end 60 of the device as the device is inserted into the body. Alternatively, removable or slidable caps or sleeves may be positioned at the distal end 60 of the device to make it easier for device insertion. A laparoscopic trocar, sleeve, lip, funnel or guide may be placed at or around the incision to allow easy insertion of the device, and this may also permit easier exchange of devices when necessary.

Referring the FIG. 3, a variation of a multi-arm robotic surgical device with an electronic interface 62 is shown. The electronic interface 62 may be provided anywhere along the distal section 64 of the device such that after the device is inserted in the patients body the interface 62 will remain outside the patient's body. In this variation, the electronic interface 62 is integrated into the proximal end 68 of the device. The interface provides electronic connections (e.g., Universal Serial Bus, serial port, or other customized connections) allowing the device to communicate with a controller. Alternatively, wireless connection such as IR communication or radio wave communications may also be implemented. The interface 62 may also have a power supply input for supplying electrical power to the various electronic components in the device body and in the robotic arms.

The controller may have a computer for controlling the surgical device such that the various components may function in a coordinated manner. Sensors and other electronic detector may also be implemented within the device to provide feedback to the controller. Furthermore, a human interface, such as a control panel with joystick or other physical interface may be provide for the surgeon to control the movements of the robotic arms directly. The surgeon's instruction may also be directed through an interface for receiving signal from the surgeon's hand (e.g., gloves with positioning sensor or tactile sensors). Alternatively, voice or other signal input mechanisms may also be used to provide the instruction. In some situation, a set of preprogrammed instructions may be executed at the command of a medical professional.

In an alternative design, the controller may be directly connected to the distal end of the surgical device. In this variation the surgeon may control the robotic arms by operating the various control interfaces on the controller that is attached to the distal end of the surgical device. For example, the surgeon may make an incision on the patient's abdomen. Insert the distal portion 66 of the surgical device into the patients body, and through the user interface and a monitor located on the controller, which is attached to the distal end of the surgical device, explore the interior of the abdomen and may additionally provide surgical intervention if necessary (e.g., operating the robotic arm to seal a ruptured vein in the abdomen).

Optionally, the device may be configured such that the distal portion 66 of the device may rotate relative to the proximal portion 64 of the device, as shown in FIG. 3. This configuration provides an additional degree of freedom in maneuvering the robotic arms located at the proximal portion 66 of the device.

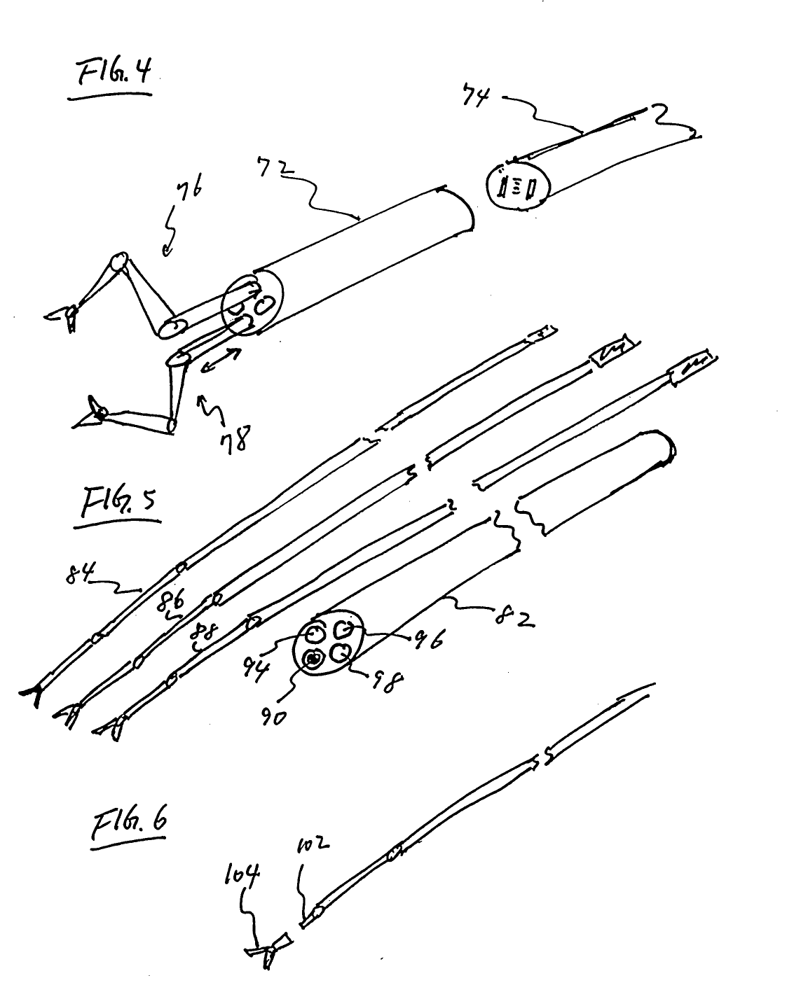

Another variation allows the surgeon to replace the distal section 72 of the robotic surgical device with new set of robotic arms that is configured for a specific surgical application, as shown in FIG. 4. This configuration allows the surgeon to switch between different sets of surgical tools during surgery without the need to provide a separate controller and other supporting electronics during the surgery. In this variation, the robotic arms 76, 78 are integrated within distal section 72 of the device.

In yet another variation as shown in FIG. 5, the device comprises a deployment conduit 82 and three separate robotic arms 84, 86, 88 that may inserted into the patients body through the deployment conduit. In this example, the deployment conduit has an integrated imaged detector 90 positioned at the distal end 92 of the deployment conduit 82. Three separate robotic arms 84, 86, 88 are inserted into the deployment conduit through ports located at the proximal section of the deployment conduit. When the user wishes to deploy the robotic arm, the user may push the robotic arms forward allowing the distal section of the robotic arm to exit the deployment conduit through ports 94, 96, 98 located at the distal end 92 of the deployment conduit 82.

To utilize the device, the surgeon may insert the deployment conduit 82 into a patient's body through an incision. Once the deployment conduit 82 is secured at the desired location, individual robotic arms 84, 86, 88 may be inserted into the deployment conduit 82. Once the robotic arm is in place, it may interlock with the deployment conduit 82, such that the distal section of the robotic arm may move in a secured manner relative to the deployment conduit. In another variation, the robotic arms 84, 86, 88 are preloaded into the deployment conduit 82. Once the deployment conduits 82 with its preloaded arms 84, 86, 88 are placed inside the patient's body, the surgeon may then deploy the robotic arms by pushing each of the robotic arm forward and extend the distal section of the robotic arm outside the deployment conduit 82.

Although, in this example, the deployment conduit provides three channels for deploying robotic arms, conduit with two, four or more channel may also be devised depending on design needs. As illustrated in FIG. 6, the distal end 102 of the individual robotic arm may have an interchange adaptor such that the surgeon may attach different surgical tools 104 to the robotic arm base on the particular need of the surgery to be performed.

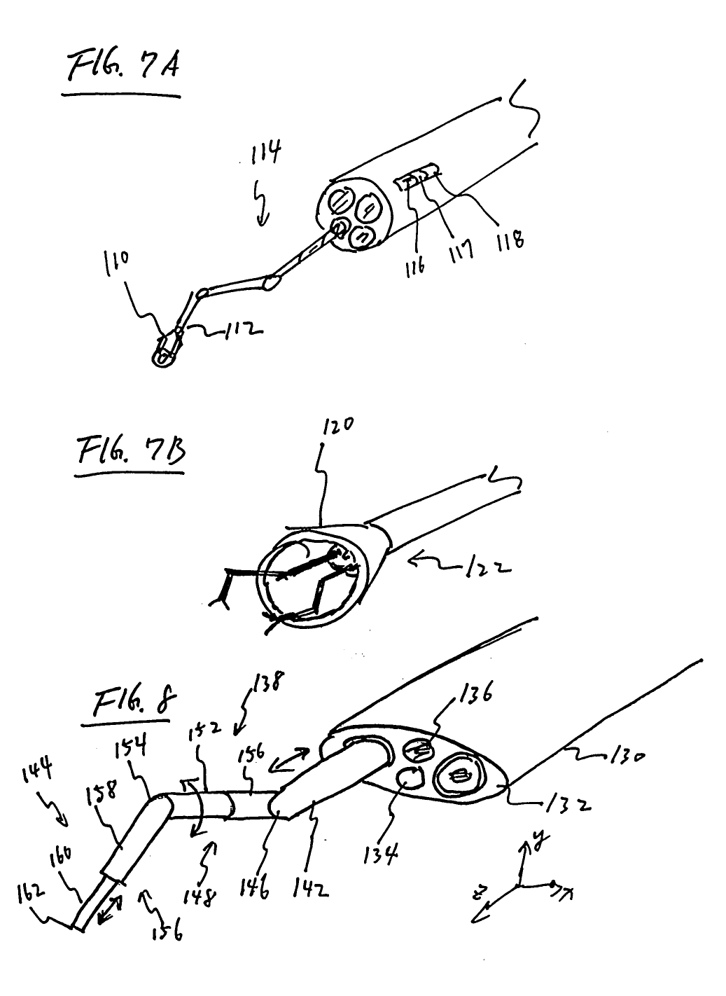

Although in the above examples, the image detector is integrated within the distal section of the device, in an alternative design, a separate robotic arm may carry the image detector to provide visual feedback. In this design variation, an integrated camera positioned on the elongated boy of the device may not be necessary. FIG. 7A shows one variation, where an image detector 110 is positioned at the distal end 112 of a robotic arm 114, and the position of the image detector may be manipulated by the user. The robotic arm 114 may carry two or more image detectors if it is desirable to capture image from more then one position simultaneously. For example, for 3D image reconstruction, two or more images may be desirable. Alternatively, image detectors may be deployed on two or more robotic arms. In addition, sensors 116, 117, 118 (e.g., pH detector, oxygen sensor, chemical sensor, Doppler sensor, temperature sensor) may be attached or integrated within the distal section of the device to monitor and provide the surgeon with information regarding the condition at the immediate area around the surgical site. Sensors may also be placed at the distal end of a robotic arm. For example, an IR detector, a chemical sensor or a Doppler sensor may be placed at the distal end 112 of a robotic arm in a similar configuration as the placement of the image detector 110 shown in FIG. 7A. In one variation, an ultrasound Doppler sensor is placed at the distal end of a robotic arm for verifying vessel patency or existence of blood flow during surgery.

Depending on the particular surgical procedure a particular multi-arm robotic surgical device may support two, three, four or more arms depending on the design criteria. Preferably, the device has a small diameter such that a small incision is enough to allow insertion of the instrument into a patient's body. Preferably, the maximal diameter (or cross-sectional width) of the portion of the device to be inserted into a patients body is 60 mm or less; more preferably, the maximal diameter is 30 mm or less; yet more preferably the maximal diameter is 20 mm or less, even more preferably the maximal diameter is 10 mm or less. In one variation, the distal portion of the device has a diameter of 12 mm, and the plurality of robotic arms are housed within individual chambers with inner diameters between 3 to 5 mm.

Furthermore, fluid suctions and fluid delivery capability may be provided within the robotic device. For example, suctions may be provide through a port located at the distal end or on the distal section of the device to remove excess fluids from the immediate area surrounding the target region for the surgery. Alternatively, the suction device may be provided through a robotic arm, such that the surgeon may remove fluids from selective area within the body cavity. A channel may be provided within the elongated body so that suction source connected to the proximal section of the device may drive a negative pressure gradient across the channel and remove liquid from the suction port located on the robotic arm or at the distal portion of the device.

A fluid delivery port may also be provided to deliver various liquids and medications to the surgical region. For example, anesthetic, muscle relaxant, vasodilator, or anticoagulant may be stored within a reservoir located within a robotic arm or within the elongated body, and ejected onto the target region through one or more ports located at the distal end or distal section of the device. Alternatively, the liquid reservoir may be connected to the proximal section of the device and a channel is provided within the elongated body to deliver the liquid to the distal section of the device.

It may also be desirable to provide a mechanism to establish a working space at the distal end of the device. For example, a port positioned at the distal section of the device may be used to provide insufflation to the cavity around the distal end of the device. A channel embedded inside the elongated body of the device may provide the path for a gas supplied at the proximal end of the device to be directed to a port at the distal end of the device. Mechanical means may also be implemented in addition to or in-place-of insufflation. For example, a conical shaped balloon 120 may be placed around the distal section 122 of the device. When the conical shaped balloon 120 is in the deflated states, it will constrict around the distal portion of the device. When the conical shaped balloon 120 is inflated, it expands both in the radial direction and in the forward direction away from the device, as shown in FIG. 7B. The expanded balloon may push the surrounding tissue away from the distal end of the device and provide a space for the robotic arms to expand and maneuver.

As one of ordinary skill in the art would appreciate, various joins and arm configurations may be implemented to provide the desired movements for the robotic arms. FIG. 8 illustrates one of the variations. In this example, the device comprise of an elongated body 130 having a oval cross-section. At the distal end 132 of the device, a light source 134 for providing illumination and an image detector 132 for providing real-time visual feedback, are integrated within the device. The device is shown with one of its arms 138 deployed. The base section 142 of the robotic arm may extend forward to provide additional reach or it may retract inward and brings the distal section 144 of the robotic arm with thin the chamber housing the robotic arm. The first primary joint 146 allow the rear-arm section 148 of the robotic arm to rotate up and down in the Y-Z plane. The rear-arm 148 comprise of a base section 150 and an rotation section 152. The rotation section 152 may rotate along the central axis of the rear-arm 148 relative the base section 150 of the rear arm. A second joint 154 is provided to allow the forearm 156 to move up and down (i.e., pitch) relative to the rear-arm 148. The forearm 156 may also comprise two sections: a base section 158 and an extendable section 160. The extendable section 160 is supported within the base section 158 and may extend and retract through actuators controlled by the user through a control interface. A tool or apparatus may be attached to the distal end 162 of the extendable section 160.

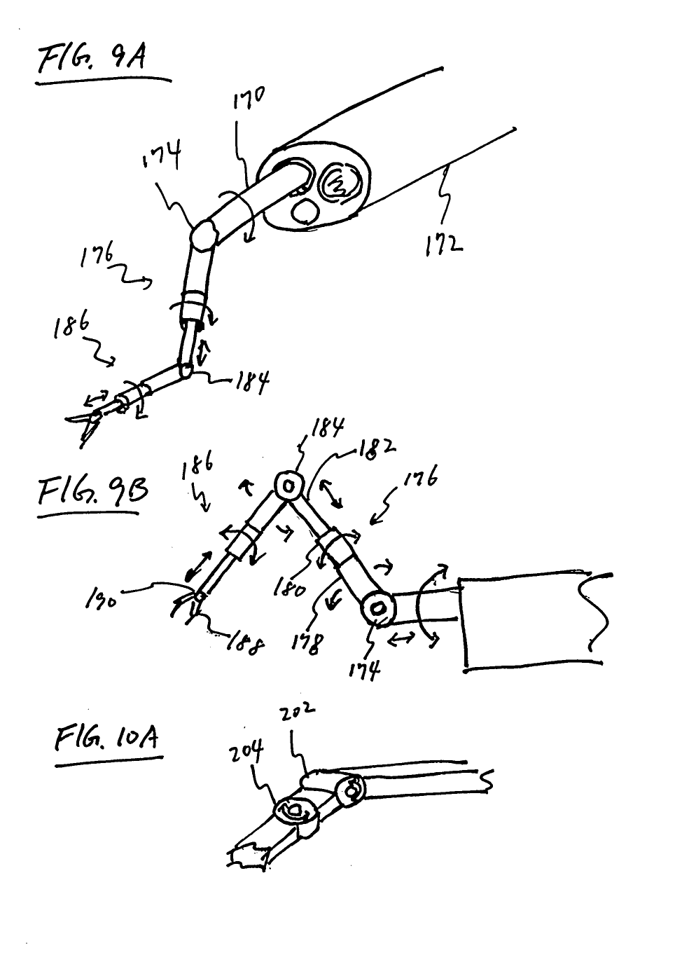

FIG. 9A shows another example of a robotic arm with improved maneuverability as compare to the example shown in FIG. 8. In this example, the base-arm section 170 may rotate along the Z-axis in relation to the elongated body 172 of the device. The shoulder joint 174 provides one degree of freedom, and allows up and down pitch motion, as shown in FIG. 9B. The rear-arm section 176 comprises three sections. The base section 178 connects to the shoulder joint 174. The rotational section 180 allows the rear-arm 176 to rotate with relation to the shoulder joint 174. An extendable section 182 is integrated within the rotational section and allows the user to extend or contract the length of the rear-arm 176. An elbow joint 184 is provided to allow the forearm 186 to move in a pitch motion relative to the rear-arm 176. The forearm 186 has a similar construction as the rear-arm 176 that allow the user to rotate and adjust the length of the arm during operation. In this variation, a clamp 188 is provided at the distal end 190 of the forearm 186 to allow the user to grasp tissues or other objects during operation. Alternatively, a joint providing two or more degree or freedom may be implemented between the clamp 188 and the distal section 190 of the forearm 186 to provide improve maneuverability to the clamp 188. As one of ordinary skill in the art would appreciate, additional joints and arm sections may be provided to extend the reach and maneuverability of the robotic arm. Various other surgical tools may also be attached to the distal end of the forearm depending on design needs.

There are various methods to implement a joint with two or more degrees of freedom, as one of ordinary skill in the art would appreciate. For example, a joint with two degrees of freedom may be accomplished by combining two rotational parts 202, 204 as shown in FIG. 10A. The first rotational part 202 provides the up-and-down movement, and the second rotational part 204 provides the right-and-left movement. Motors may be built into each of the rotational parts to drive the motion of the attached arm. Controller directs electrical current to the embedded motor and drive the motor to produce the desired motion.

FIG. 10B illustrates an example implementing a joint with two degrees of freedom. The first joint 210 comprised of a first rotational part 212 to provide the up-and-down motion (i.e., pitch), and the second rotational part 214 provides the right-and-left motion (i.e., yaw). In combination, they provide the rear-arm 216 with two degrees of freedom. The second joint 218 is comprises a signal rotational part to provide only the up-and-down motion. The distal end 220 of the forearm 222 comprises an adapter for receiving different surgical tools 224.

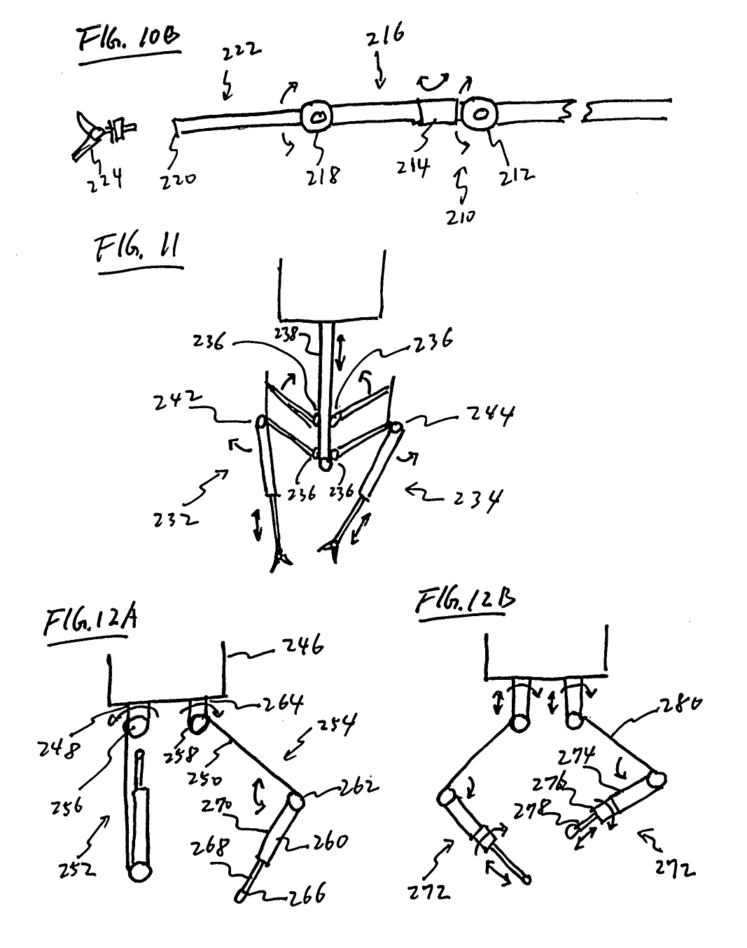

As one of ordinary skill in the art would appreciate, other configurations may also be implemented to provide lateral expansion of the robotic arms. For example, as shown in FIG. 11, a laterally expendable skeleton may be provided to deploy the robotic arms 232, 234 after the device is inserted inside the patient's body. Joints 236 attached to a central bar 238 allow the frames supporting the robotic arms to flare outwards, positioning the primary joints 242, 244 of the robotic arms away from the central bar. These primary joints 242, 244 allow the sections of the arms 232, 234 that are connected to them to rotate inward and directing the distal section of the arm toward a target region. In this example, the arms 232, 234 are configured such that they may extend and contract as needed. Additional joints may be implemented on the arm to provide additional degrees of freedom.

Various approaches may be implemented to store the robotic arms in a compact configuration for easy insertion into the patient's body. After the robotic arms are positioned within the body they may be deployed to accomplish the prescribed surgical task. FIG. 12A illustrates one approach to store robotic arms in a confined space. The two robotic arms 252, 254 are stored within chambers located within the distal portion 246 of an elongated tube. For deployment, the base of the arm 248 is pushed forward by an actuator, allowing the rear-arm 250 and forearm 260 sections of the robotic arm expose outside the chamber. The base of the arm may rotate (along the axis extending into the length of the tube) relative the elongated tube, and as the result, rotate the complete arm. The right arm 252 is shown in a closed position, and the left arm 254 is shown in an opened position. The shoulder joint 256, 258 may rotate and allow the rear-arm to rotate outward in the lateral direction. Various mechanisms well know to one of ordinary skill in the art may be implemented to drive the rotation of the arm about the joint. For example, a motor may be embedded inside the joint to drive the rotational motion. Alternatively, a motor may be placed inside the base of the arm 248 to drive the rotation of the rear-arm 252. As shown in FIG. 12A, the forearm 260 may be folded back on top of the rear-arm 250. The elbow joint 262 allow the forearm to rotate outward to the deployed position as illustrated by the right arm 254 in FIG. 12A. A motor may be position inside the elbow joint 262 to drive the rotation of the forearm 260 in relation to the rear-arm 250. By controlling the rotation of the base 264, the rotation of the shoulder joint 258 and the rotation of the elbow joint 262, the operator may direct the distal end 266 of the forearm to a desired location. To provide additional reach, the forearm 260 is configured with two sections. The front section 268 is placed inside the back section 270 and may be displaced using an actuator or motor. The operator, by controlling the electrical current supplied to the motor may extend or retract the front section 268 of the forearm 260 as desired.

In another variation of the device shown in FIG. 12A, the forearm 272 is configured with the additional capability to rotate along the central axis parallel to the length of the forearm 272, as shown in FIG. 12B. This axial rotation provides an additional degree of freedom for maneuvering a device or tool connected to the distal end of the forearm. The base section 274 and the midsection 276 of the forearm are interlinked and may rotate relative to each other. The distal section 278 of the forearm is connected to the mid-section 276 and may extend outward from the mid section 276. A motor may be position within the base section to drive the midsection of the forearm. As the midsection of the forearm rotates, the distal section 278 and any tools attached to the distal section 278 would also rotate. This configuration may allow easy deployment of the robotic arms in a confined space. In particular, the rear-arm 280 may expand radially and push aside tissues around the distal end of the device to provide a working space for the robotic arms.

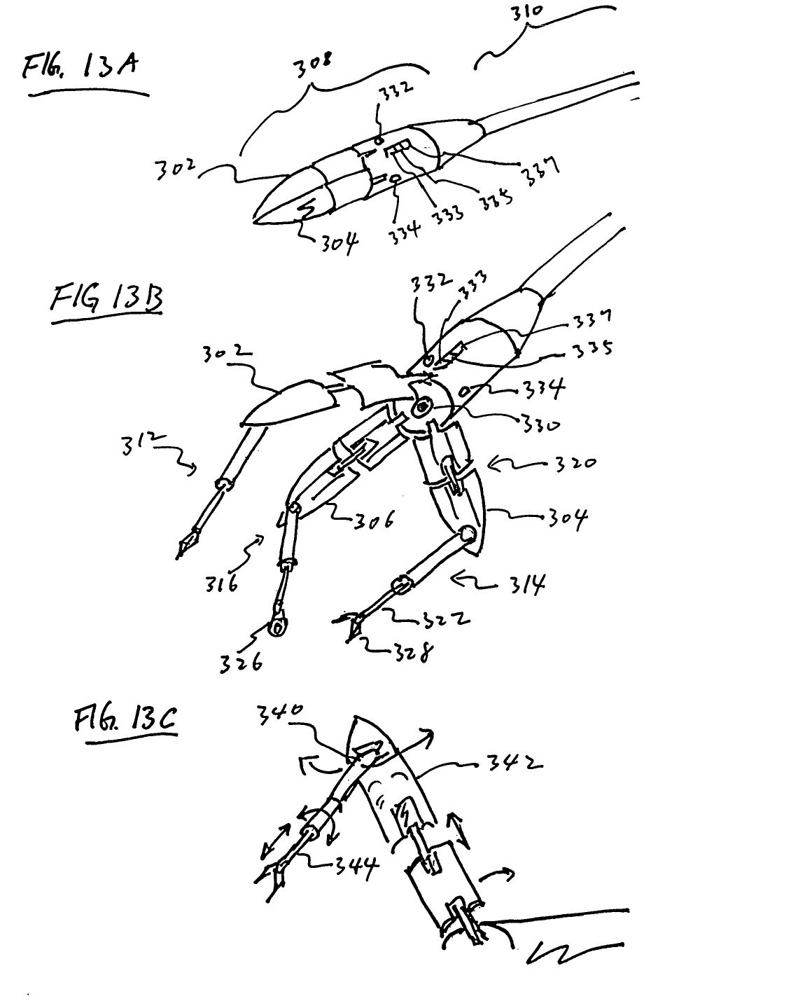

In an alternative design, the device's distal end May comprise a plurality of leaflets. The device with its leaflets 302, 304, 306 in the closed position, as shown in FIG. 13A, allows easy insertion of the device into a patient's body. The distal portion 306 of the device may have a larger diameter than the proximal portion 208 of the device. This design may allow insertion of a device having a large diameter distal portion through a small hole by temporally stretching the hole so that the distal portion 308 may pass through, but since the proximal portion 310 of the device has smaller diameter it will not stress the orifice after the distal portion of the device has been inserted into the body.

For deployment of the robotic arms, the leaflets 302, 303, 304 expands radially and exposes the robotic arms, as shown in FIG. 13B. In this variation, the each of the robotic arms 312, 314, 316 are attached to the distal ends of the leaflets 302, 204, 306 through a joint. A displacement interface 320 is provided at the midsection of each leaflet so that the leaflets may expand longitudinally. Motors or actuators may be implemented inside the body of the device to control the angel of the leaflets as they are opened up. Each of the robotic arms 312, 314, 16 has an extension section 322 that may be extended or retracted to change the reach of the arm. In addition, each of the arms may rotate along the longitudinal axis along the length of the arm. One of the arms is shown with a blade 324 at its distal end, the second arm has a camera 326, and the third arm has a forceps 328. Optionally, a camera 330 may be provided at the interface region where the leaflets connect to the body of the device, as shown in FIG. 13B. On the body of the device, a port 332 may be provided for infusing gas into the patient's body to provide insufflation. A channel may be built into the device to direct fluid flow from the proximal end of the device to the port. In addition, a port 334 may be provided to remove liquid from the area surrounding distal portion of the device. An internal channel connected to a suction device may be utilized to generate a negative pressure region around the suction port 334. Furthermore, temperature and chemical sensors 333, 335, 337 may be provided on the body of the device for measuring the temperature and chemicals inside the patient's body.

In another variation, the base of the arm 340 may rotate from side to side (i.e., laterally relative to the length of the leaflet) relative to the leaflet 342 that supports it, as illustrated in FIG. 13C. In addition, the robotic arm can rotate along the long axis of the arm and the distal portion 344 of the arm is retractable.

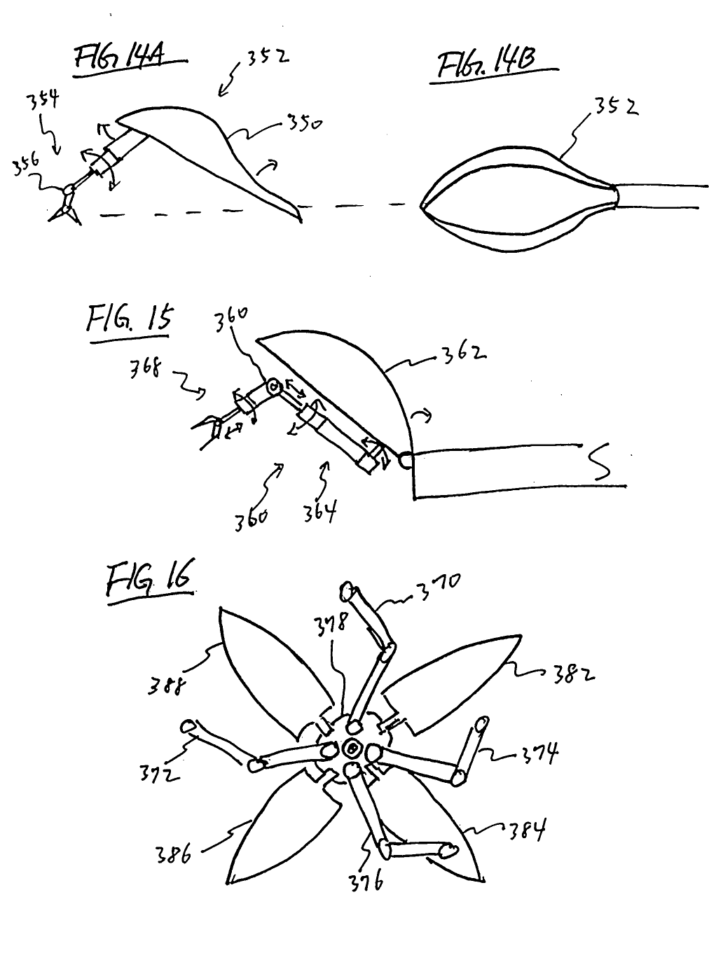

Referring to FIG. 14A, in this variation, the leaflet 350 is designed with a dome shape at the distal portion 352 of the leaflet to provide room to house a robotic arm under the leaflet. This design may allow larger and more complex mechanical arm 354 be implemented under the leaflets, as shown in FIG. 14B. Optionally, an additional joint 356 may be provided at the distal end of the arm so that the tools connected at the distal end of the arm may have two or more degrees of freedom.

FIG. 15 illustrates anther variation where the robotic arm is connected to the based of the leaflet. In this design, the robotic arm 360 may rotate at the based of the arm relative to the leaflet 362. A rear-arm section 364 is provided with both extension/retraction capability and the ability to rotate along the axis of the rear-arm. A joint 366 is provided between the forearm 368 and the rear-arm 364 to allow the forearm 368 to rotate relative to the rear-arm. The forearm 368 is also provided with the ability to both the capability to extend/retract and rotate along the axis of the forearm.

In yet another variation, the robotic arms 370, 372, 374, 376 connected to the main body 378 of the device, as shown in FIG. 16. Once the distal portion of the device is inserted inside the patient's body, the leaflets 382, 384, 386, 388 are opened up and the surgeon may maneuver the various arms 372, 374 376, 378 to complete the necessary surgical task. In this example, after the procedure is completed, the arms 372, 374, 376, 378 collapses into the center of the device and the leaflets 382, 384, 386 388 closes over them and covers the robotic arms to allow easy removal of the device form the body. In an alternative design. The robotic arms are housed within the body of the device and the leaflets at the distal end of the device are provided to cover the distal end of the device and to provide a tapered head region for easy insertion. Once the device in inserted, the leaflets open up to allow the robotic arms to extend out of the chambers housing the robotic arms. Once the surgery is completed, the arms are retracted back into the chambers and the leaflets are closed before the device is removed from the patient's body.

The device describe herein may be implemented to perform various minimally invasive surgical procedures. For example, one approach for performing a cholecystectomy with a multi-arm robotic surgical device is described below. The surgeon first makes an incision around the umbilical area for insertion of the device through the skin and muscle tissues into the abdominal cavity. Than a needle is used to insufflate the abdomen. After satisfactory insufflation, the distal portion of the device is inserted into the patient's abdomen. With the assistance of the image detector located at the distal end of the device, the surgeon then maneuvers the device into position so that the gallbladder is visible through the image detector. A holder or rack may be attached to the proximal portion of the device to secure the device in position. The three robotic arms are then deployed at the distal end of the device. The first arm has a bipolar forceps connected to the distal end of the robotic arm. The second arm has a scissor connected to the distal end of the robotic arm. And the third arm has a vascular clip applicator attached to the distal end of the robotic arm.

The surgeon first dissects some of the tissues surrounding the gallbladder with the forceps and the scissor to expose the cystic duct and the cystic artery. Electric current may be directed down the bipolar forceps to seal off any blood vessels to prevent bleeding. The cystic duct is then dissected free. Vascular clip applicator applied to seal of the cystic duct. The cystic duct is then transected using the scissor. Next, the bipolar forceps and the scissors are used again to dissect free the cystic artery. The vascular clip applicator applied again to seal of the cystic artery. The surgeon then dissects the gallbladder off the liver bed with the bipolar forceps and the scissor. The gallbladder may then be removed from the patient's body.

In anther example, the multi-arm surgical device is used to perform an appendectomy. A small incision is made on the patient's abdomen, followed by insufflation of the abdomen. The distal portion of a multi-arm surgical device is then inserted into the patient's abdomen. Preferably, the size of the device is small enough that it will fit through an incision with a width of 60 millimeters or smaller. More preferable, the incision has a width that is 40 millimeters or smaller. Yet more preferably, the incision has a width of 30 millimeters or smaller. Even more preferably, the incision has a width of 20 millimeters or smaller.

The distal end of the device is positioned above the appendix so the surgeon may inspect the appendix. Through maneuvering a bipolar forceps on the first robotic arm and a scissor on the second robotic arm, the surgeon first free up the appendix from the large bowel which the appendix is attached. This requires dividing the mesentery which contains the blood vessels that supply the appendix. The bipolar forceps is used to apply electric current and seal off the blood vessels, and scissors are used at the same time to divide the mesentery. By applying the bipolar forceps and the scissors in a coordinated manner through the robotic arms, the appendix is completely mobilized down to its base. The third robotic arm carrying a pre-tied suture is then deployed. With the assistance of the bipolar forceps, the suture is placed around the neck of the appendix and then tightened. Excess sutures are then cut with the scissors. Finally, with the bipolar forceps holding on to the neck of the appendix, the scissor is used to cut free the appendix. The appendix is then ready to be removed.

All publications and patent applications cited in this specification are herein incorporated by reference in their entirety as if each individual publication or patent application were specifically and individually put forth in the text below.

This invention has been described and specific examples of the invention have been portrayed. While the invention has been described in terms of particular variations and illustrative figures, those of ordinary skill in the art will recognize that the invention is not limited to the variations or figures described. In addition, where methods and steps described above indicate certain events occurring in certain order, those of ordinary skill in the art will recognize that the ordering of certain steps may be modified and that such modifications are in accordance with the variations of the invention. Additionally, certain of the steps may be performed concurrently in a parallel process when possible, as well as performed sequentially as described above.

Therefore, to the extent there are variations of the invention, which are within the spirit of the disclosure or equivalent to the inventions found in the claims, it is my intent that this patent will cover those variations as well.

1. A robotic surgical device comprising:
an elongated body; and
a plurality of robotic arms extendable from a distal portion of said elongate body, wherein at least one of said robotic arms comprises two or more joints.

2. The robotic surgical device of claim 1 further comprising:
an image detector positioned at said distal portion of the said elongated body.

3. The robotic surgical device of claim 1 wherein said robotic arms are housed within said distal portion of said elongated body and each of said robotic arms is further configured for deployment through a distal end of said elongated body.

4. The robotic surgical device of claim 1 wherein at least one of said robotic arms is housed within a separate chamber located within said distal portion of said elongated body and each of said chambers has a port located at a distal end of said elongated body for deployment of said robotic arm.

5. The robotic surgical device of claim 1 wherein at least one of said robotic arms further comprises a surgical tool attached to a distal end of said robotic arm.

6. The robotic surgical device of claim 1 wherein at lease two of said robotic arms comprise a rear-arm with a proximal end connected to said elongated body through a first joint, and a forearm connected to a distal end of said rear-arm through a second joint, wherein said first joint permits a distal end of said rear-arms to expand radially from a center axis of said elongated body.

7. The robotic surgical device of claim 6 wherein said second joint permits a distal end of said forearm to converge toward said central axis of said elongated body while said rear-arm is expanded radially.

8. The robotic surgical device of claim 7 wherein each of said robotic arms further comprises a surgical tool attached to a distal end of said robotic arm.

9. The robotic surgical device of claim 2 wherein said image detector is attached to a distal end of said elongated body.

10. The robotic surgical device of claim 1 further comprising:
an image detector attached to one of said robotic arms.

11. A robotic surgical device for performing minimally invasive surgery comprising:
an elongated tubular body having a plurality of chambers, each of said chambers has an opening at the distal end of said elongated tubular body; and
a plurality of robotic arms, wherein each of said robotic arms is slideably positioned within one of said chambers.

12. The robotic surgical device of claim 11 further comprising:
a camera attached to said distal end of said elongated tubular body.

13. The robotic surgical device of claim 11 wherein a distal portion of said elongated tubular body has a diameter of 30 millimeter or less.

14. The robotic surgical device of claim 11 comprising three or more robotic arms.

15. The robotic surgical device of claim 11 wherein each of said robotic arms comprises at least three arm sections, a fist arm section slidably adapted within one of said chamber, said first arm section connects to a second arm section through a fist joint, and a second arm section connected to a third arm section through a second joint, wherein said first joint allows said second arm section to rotate relative to said first arm section while at the same time the third arm section can rotates about the second joint in a direction independent of the rotation of said second arm section.

16. The robotic surgical device of claim 15 further comprising:
a camera attached to said distal end of said elongated tubular body.

17. The robotic surgical device of claim 16 wherein each of said robotic arms further comprises a surgical tool connected to a distal end of said third arm section.

18. The robotic surgical device of claim 17 wherein a distal portion of said elongated tubular body has a diameter of 12 millimeter or less, and each of said robotic arms has a diameter of 5 millimeter or less.

19. The robotic surgical device of claim 16 wherein each of said robotic arms further comprises a third joint having at least three degrees of freedom connected to a distal end of said third arm section, and a surgical tool is connected to said third joint.

20. The robotic surgical device of claim 19 wherein said robotic arms are configured such that as least two robotic arms can be directed by a user to approach a predefined tissue region from two separate directions.

21. A method for performing a minimally invasive surgical procedure comprising:
inserting a distal portion of an elongated robotic surgical device into a patient's body; and
deploying a plurality of robotic arms through a distal end of said robotic surgical device.

22. The method of claim 21 further comprising:
operating said robotic arms through visual feedbacks provided by an image detector positioned at a distal end of said robotic surgical device.

23. The method of claim 22 further comprising:
operating two or more of said robotic arms to dissect tissues within said patients body.

24. The method of claim 21 further comprising:
making an incision on said patient's body prior to inserting said distal section of said elongated device into said patients body through said incision, wherein said incision has a width of less than thirty millimeters.

25. The method of claim 21 wherein each of said robotic arms comprises two or more joints.

26. The method of claim 21 wherein each of said robotic arms comprises a rear-arm connected to said distal section of said robotic device through a shoulder joint, and a forearm connected to said rear-arm through an elbow joint.

27. The method of claim 23 further comprising the step of:
rotating said rear-arm away from a central a axis of said elongated robotic surgical device while at the same time rotating said forearm toward said central axis.

28. The method of claim 21 further comprising the step of:
maneuvering said robotic arms to detach said patient's gallbladder from tissues surrounding said gallbladder.

29. The method of claim 21 further comprising the step of:
maneuvering at least two of said robotic arms simultaneously in a coordinated manner inside said patient's body.
